(19) United States
(12) Patent Application Publication
(10) Pub. No.: US 2021/0299103 A1
(43) Pub. Date: Sep. 30, 2021

(54) COMPOSITION FOR PREVENTING OR TREATING CANCER, COMPRISING SULFONAMIDE DERIVATIVE AS ACTIVE INGREDIENT

(71) Applicant: EXOSTEMTECH CO., LTD., Ansan (KR)

(72) Inventors: Moon-Chang BAEK, Daegu (KR); Eun-Ju IM, Daegu (KR)

(21) Appl. No.: 17/264,692

(22) PCT Filed: Jul. 26, 2019
(86) PCT No.: PCT/KR2019/009359

§ 371 Date: Jan. 29, 2021

(30) Foreign Application Priority Data
Aug. 1, 2018 (KR) 10-2018-0089653

(51) Int. Cl. A61K 31/42; A23L 33/00; A23L 29/00; A61P 35/04; A61P 31/04

(52) U.S. Cl. CPC A61K 31/42; A23L 33/30; A61P 31/04; A61P 35/04; A23L 29/055

(57) ABSTRACT

The present disclosure relates to a composition for treating cancer and inhibiting cancer metastasis, which contains a sulfonamide derivative as an active ingredient. More specifically, the composition containing a sulfonamide derivative as an active ingredient may be provided as a cancer therapeutic agent or a cancer metastasis inhibitor since the sulfonamide derivative from which antibiotic susceptibility has been completely removed exhibits superior effect of inhibiting cancer cell multiplication and migration by effectively inhibiting the secretion of exosomes by cancer cells and exhibits no cytotoxicity to normal cells.

### TECHNICAL FIELD

The present disclosure relates to a composition for treating cancer and inhibiting cancer metastasis, which contains a sulfonamide derivative as an active ingredient.

### BACKGROUND ART

Cancer is one of incurable diseases that should be overcome by the humanity. Globally, a large amount of capital is invested for the development of the cure of this disease. Since 1983, cancer has been the number one cause of death in Korea. About 100,000 people are diagnosed with cancer and about 60,000 people die from it every year.

The causes of cancer include smoking, ultraviolet radiation, chemicals, foods and other environmental factors. Due to the various causes, the development of therapeutic agents is difficult and the effect of the therapeutic agents is different depending on the sites of onset.

Currently used anticancer agents include biological agents such as enzyme agent, vaccines, etc., synthetic medications, naturally derived medications, etc. Among them, anticancer agents using genes, enzymes, vaccines, etc. are not commercially available yet, and chemotherapeutic anticancer agents have significant toxicity and have side effects that cancer cells are not selectively removed but normal cells, especially actively dividing cells, are also affected. In addition, the chemotherapeutic anticancer agents are not effective for cancer treatment due to the resistance of cancer cells. Accordingly, for treatment and prevention of cancer, development of an effective anticancer agent which has little toxicity and does not cause resistance of cancer cells is urgently needed.

Meanwhile, exosomes with sizes of 50-200 nm are secreted by almost all the type of cells. In particular, the exosomes secreted from cancer cells are known to affect cancer metastasis, angiogenesis and cancer cell multiplication during the progression of cancer. Since cancer cell multiplication and metastasis can be inhibited by reducing the role of exosomes by inhibiting the secretion of exosomes from cancer cells during the progression of cancer, research and development of a new-concept anticancer agent which inhibits the secretion of exosomes from cancer cells are necessary.

### DISCLOSURE

### Technical Problem

The present disclosure is directed to providing a composition containing a sulfonamide derivative, which inhibits antibiotic effect and exhibits an effect of inhibiting secretion of exosomes from cancer cells, as an active ingredient, as a composition for treating cancer and inhibiting cancer metastasis, in order to solve the problem of the side effect of a chemotherapeutic agent which exhibits cytotoxicity to normal cells other than malignant tumors.

### Technical Solution

The present disclosure provides a pharmaceutical composition for preventing or treating cancer, which contains a compound represented by Chemical Formula 1 or a pharmaceutically acceptable salt thereof as an active ingredient.

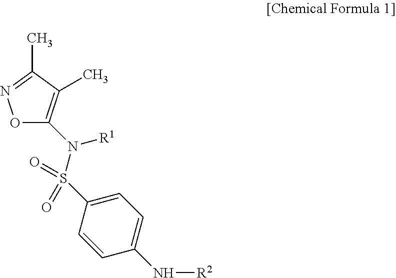

In Chemical Formula 1,

one of R¹ and R² is acetyl, and the other is any of hydrogen or acetyl.

The present disclosure provides a health food (food supplement) for preventing or improving cancer, which contains the compound represented by Chemical Formula 1 or a pharmaceutically acceptable salt thereof as an active ingredient.

The present disclosure provides a pharmaceutical composition for inhibiting cancer metastasis, which contains the compound represented by Chemical Formula 1 or a pharmaceutically acceptable salt thereof as an active ingredient.

In addition, the present disclosure provides a health food for inhibiting cancer metastasis, which contains the compound represented by Chemical Formula 1 or a pharmaceutically acceptable salt thereof as an active ingredient.

### Advantageous Effects

According to the present disclosure, a composition containing a sulfonamide derivative as an active ingredient may be provided as a cancer therapeutic agent or a cancer metastasis inhibitor since the sulfonamide derivative from which antibiotic susceptibility has been completely removed exhibits superior effect of inhibiting cancer cell multiplication and migration by effectively inhibiting the secretion of exosomes by cancer cells and exhibits no cytotoxicity to normal cells.

### DESCRIPTION OF DRAWINGS

FIG. 1 shows a reaction scheme for synthesis of a sulfisoxazole derivative.

FIG. 2 shows the chemical structure of sulfisoxazole (SFX-WT) and its derivatives, sulfisoxazole-N¹-acetyl (N1AS), sulfisoxazole-N⁴-acetyl (N4AS) and sulfisoxazole-N¹&N⁴-dual acetyl (DAS).

FIG. 3 shows a result of investigating the cytotoxicity of SFX-WT, N1AS, N4AS and DAS in MDA-MB231 breast cancer cells.

FIG. 4 shows a result of investigating the exosome secretion-inhibiting effect of SFX-WT, N1AS, N4AS and DAS in MDA-MB231 breast cancer cells.

FIG. 5 shows a result of investigating the cancer cell migration-inhibiting effect of SFX-WT, N1AS, N4AS and DAS in MDA-MB231 breast cancer cells.

### BEST MODE

Hereinafter, the present disclosure is described in more detail.

The chemotherapeutic agents currently used for treatment of malignant tumors have significant side effects because they affect normal cells, especially actively dividing cells, rather than acting selectively on cancer cells only. Therefore, the inventors of the present disclosure have studied on a new-concept anticancer agent. In doing so, they have identified that a sulfonamide derivative exhibits superior anticancer effect by effectively inhibiting the secretion of exosomes from cancer cells and is safe for normal cells and completed the present disclosure.

The present disclosure may provide a pharmaceutical composition for preventing or treating cancer, which contains a compound represented by Chemical Formula 1 or a pharmaceutically acceptable salt thereof as an active ingredient.

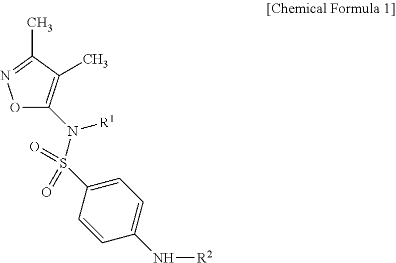

In Chemical Formula 1,

one of R¹ and R² is acetyl, and the other is any of hydrogen or acetyl.

More specifically, the compound may be selected from a group consisting of sulfisoxazole-N¹-acetyl, sulfisoxazole-N⁴-acetyl and sulfisoxazole-N¹&N⁴-dual acetyl.

The compound or a pharmaceutically acceptable salt thereof may inhibit the growth and metastasis of cancer cells by inhibiting the secretion of exosomes while inhibiting antibiotic effect.

According to examples of the present disclosure, as a result of investigating minimum inhibitory concentrations (MICs) against Staphylococcus aureus (ATCC 29213) and Escherichia coli (ATCC 25922) for identification of the antibiotic susceptibility of sulfisoxazole derivatives, it was confirmed that, whereas the sulfisoxazole (SFX-WT) compound exhibits strong antibiotic susceptibility for S. aureus and E. coli, the sulfisoxazole derivative SFX-N1AS shows decreased antibiotic susceptibility and, especially, SFX-N4AS and SFX-DAS show no antibiotic susceptibility at all, as described in Table 1.

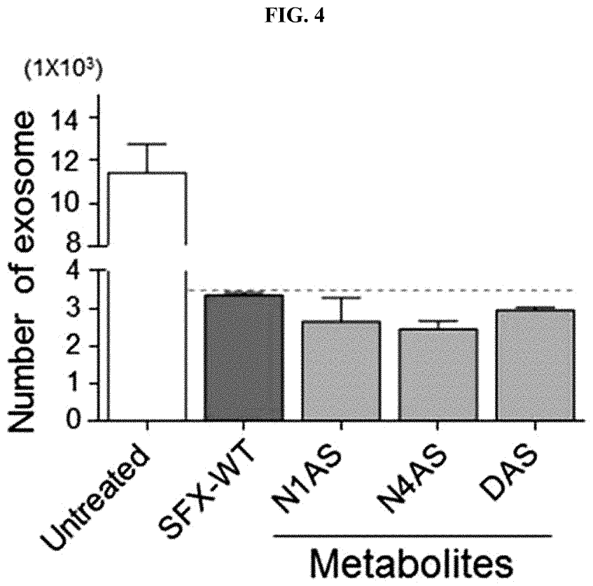

In addition, as a result of treating MDA-MB231 breast cancer cells with 100 μM of each sulfisoxazole derivative and measuring the secretion of exosomes from the cancer cells after culturing for 24 hours, it was confirmed that all of N1AS, N4AS and DAS strongly inhibit the secretion of exosomes although antibiotic effect was completely lost, as shown in FIG. 4.

Based on these results, the sulfisoxazole derivative of the present disclosure can be a new anticancer therapeutic agent capable of perfectly solving the side effect problem due to the antibiotic effect of sulfisoxazole, which may affect normal cells.

The cancer may be selected from a group consisting of non-small-cell lung cancer, breast cancer, ovarian cancer, uterine cancer, pancreatic cancer, lung cancer, stomach cancer, liver cancer, colon cancer, skin cancer, head and neck cancer, brain cancer, laryngeal cancer, prostate cancer, bladder cancer, esophageal cancer, thyroid cancer, kidney cancer, rectal cancer, acute myeloid leukemia, chronic myeloid leukemia, acute lymphocytic leukemia, chronic lymphocytic leukemia and blood cancer. More specifically, it may be breast cancer, further more specifically, triple-negative breast cancer, although not being limited thereto.

In addition, the present disclosure may provide a health food for preventing or improving cancer, which contains a compound represented by Chemical Formula 1 or a pharmaceutically acceptable salt thereof as an active ingredient.

The present disclosure may provide a pharmaceutical composition for inhibiting cancer metastasis, which contains a compound represented by Chemical Formula 1 or a pharmaceutically acceptable salt thereof as an active ingredient.

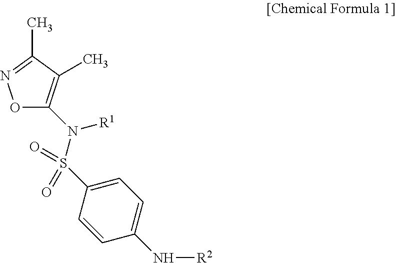

In Chemical Formula 1,

one of R¹ and R² is acetyl, and the other is any of hydrogen or acetyl.

In addition, the present disclosure may provide a health food for inhibiting cancer metastasis, which contains the compound represented by Chemical Formula 1 or a pharmaceutically acceptable salt thereof as an active ingredient.

In the present disclosure, the cancer metastasis refers to formation of new cancer due to spread of cancer cells from a primary site to other organs. The prevention or regulation of metastasis is a major target in cancer research because it is an important phenomenon that threatens the lives of patients with various cancers. Although surgery, anticancer therapy or radiation therapy is effective if cancer is diagnosed in the early stage before metastasis, the therapeutic effect is decreased after metastasis has occurred. Often, metastasis, which has not been found upon diagnosis, is found during or after treatment. Although cancer metastasis is clinically very important, the process of metastasis is not fully understood yet.

Metastasis consists of continuous stages such as invasion, intravasation, arrest, extravasation, colonization, etc. Through these processes, cancer spreads from the primary organ to other organs. Invasion, which is the first step of metastasis, involves change in interactions between cancer cells or with the extracellular matrix, degradation of surrounding tissues, migration of cancer cells into tissues, etc.

In the second step, intravasation, cancer cells enter the systemic circulation through blood or lymphatic vessels. It is reported that only a fractional portion of the intravasating cancer cells survive during circulation, and some of the surviving cancer cells successfully penetrate into capillary endothelial cells of other sites through extravasation and adapt to a new environment, forming metastatic cancer.

In an example of the present disclosure, the effect of inhibiting cancer cell invasion was investigated by Matrigel invasion assay. After placing an insert with a pore size of 8 μm on a 24-well plate and adding 600 μL of 1% FBS to a lower chamber and 1×10⁴ MDA-MB231 cells to an upper chamber, the upper chamber was treated with SFX-WT, SFX-N1AS, SFX-N4AS or SFX DAS at 100, 200, 400 or 800 μM. 5 hours later, the insert was removed and the lower portion of the chamber was fixed with 60% ethanol and stained with crystal violet.

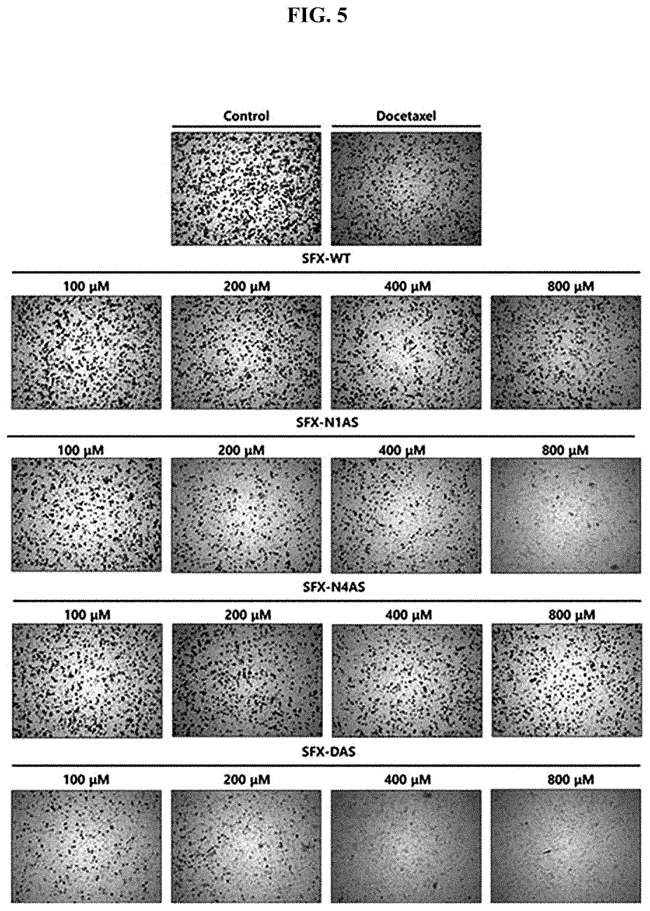

As a result, whereas a lot of cancer cell migrated to the center in the control group, the migration of the cancer cells was inhibited in a concentration-dependent manner in the groups treated with SFX-WT, SFX-N1AS, SFX-N4AS or SFX-DAS, as shown in FIG. 5.

The pharmaceutical composition according to the present disclosure may further contain a suitable carrier, excipient or diluent commonly used in preparation of pharmaceutical compositions.

Examples of the carrier, excipient or diluent that may be used in the present disclosure include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc.

The pharmaceutical composition according to the present disclosure may be formulated into an oral formulation such as a powder, a granule, a tablet, a capsule, a suspension, an emulsion, a syrup, an aerosol, etc., a formulation for external application, a suppository or a sterile injection solution according to common methods.

The formulation is prepared using a commonly used diluent or excipient such as a filler, an extender, a binder, a wetting agent, a disintegrant, a surfactant, etc. Solid formulations for oral administration include a tablet, a pill, a powder, a granule, a capsule, etc., and the solid formulation may be prepared by mixing the above-described compound with at least one excipient, e.g., starch, calcium carbonate, sucrose, lactose, gelatin, etc.

In addition to the simple excipients, a lubricant such as magnesium stearate or talc is also used. Liquid formulations for oral administration include a suspension, a liquid for internal use, an emulsion, a syrup, etc. The liquid formulation may contain, in addition to a commonly used simple diluent such as water or liquid paraffin, various excipients, e.g., a wetting agent, a sweetener, an aromatic, a preservative, etc.

Formulations for parenteral administration include a sterilized aqueous solution, a non-aqueous solution, a suspension, an emulsion, a lyophilized formulation and a suppository. As the non-aqueous solution or the suspension, propylene glycol, polyethylene glycol, a vegetable oil such as olive oil, an injectable ester such as ethyl oleate, etc. may be used. As a base of the suppository, Witepsol, macrogol, Tween 61, cocoa butter, laurin butter, glycerogelatin, etc. may be used.

And, the administration dosage of the pharmaceutical composition according to the present disclosure may be increased or decreased depending on administration route, disease severity, sex, body weight, age, etc. Accordingly, the administration dosage does not limit the scope of the present disclosure by any means.

The pharmaceutical composition may be administered to mammals such as rat, mouse, livestock, human, etc. via various routes. All modes of administration may be expected. For example, the administration may be orally, intrarectally, intravenously, intramuscularly, subcutaneously, intratracheally, intrauterinely or intracerebroventricularly.

A functional health food (food supplement) may be provided into form of a powder, a granule, a tablet, a capsule, a syrup or a drink. The functional health food which contains the compound represented by Chemical Formula 1 as an active ingredient may be used together with other foods or food ingredients according to common methods. The mixing amount of the active ingredient may be determined adequately depending on the purpose of use, e.g., prevention, health improvement or therapeutic treatment.

The amount of the compound represented by Chemical Formula 1 contained in the functional health food according to the present disclosure may be determined in accordance with the effective dose of the pharmaceutical composition. However, in case of long-term intake for the purpose of health or hygiene improvement, the amount may be less than the above-described range. In addition, the amount may also be more than the above-described range because the active ingredient has no problem in terms of safety.

The type of the functional health food is not specially limited. Examples include meat, sausage, bread, chocolate, candy, snack, confectionery, pizza, ramyeon, other noodles, gums, dairy products including ice creams, soups, beverages, tea, drinks, alcoholic beverages, vitamin complexes, etc.

The compound represented by Chemical Formula 1 of the present disclosure includes, not only the pharmaceutically acceptable salt, but also all salts, hydrates and solvates that may be prepared by common methods.

An addition salt according to the present disclosure may be prepared by common methods. For example, it may be prepared by dissolving the compound of Chemical Formula 1 in a water-soluble organic solvent, e.g., acetone, methanol, ethanol, acetonitrile, etc. and adding an excessive amount of an organic acid, or by adding the compound to an aqueous solution of an inorganic acid and conducting precipitation or crystallization. Then, after evaporating the solvent or excess acid from the mixture, the addition salt may be obtained by drying or suction-filtering the precipitated salt.

In addition, the present disclosure may provide a reagent composition for inhibiting the secretion of exosomes, which contains the compound represented by Chemical Formula 1 or a pharmaceutically acceptable salt thereof as an active ingredient, wherein the compound or a pharmaceutically acceptable salt thereof inhibits antibiotic effect in vitro and inhibits the secretion of exosomes from cancer cells.

In addition, the present disclosure may provide a method for inhibiting the secretion of exosomes while inhibiting antibiotic effect in vitro, which includes a step of treating cancer cells isolated from a non-human subject with the compound represented by Chemical Formula 1 or a pharmaceutically acceptable salt thereof.

### Mode for Disclosure

Hereinafter, the present disclosure will be described in more detail through examples to help understanding of the present disclosure. However, the following examples merely exemplify the present disclosure and the scope of the present disclosure is not limited by the examples. The examples of the present disclosure are provided to more fully describe the present disclosure to those having ordinary knowledge in the art.

### <Example 1> Synthesis of Sulfisoxazole Derivatives

Sulfisoxazole (SFX) was purchased from Sigma (31739) and sulfisoxazole-N¹-acetyl (N1AS), sulfisoxazole-N⁴-acetyl (N4AS) and sulfisoxazole-N¹&N⁴-dual acetyl (DAS), which are metabolites of SFX, were synthesized in the laboratory.

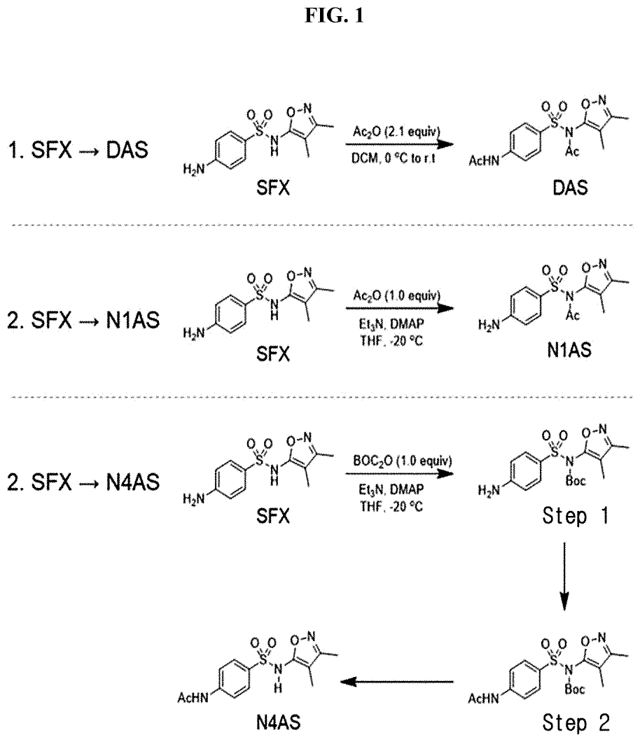

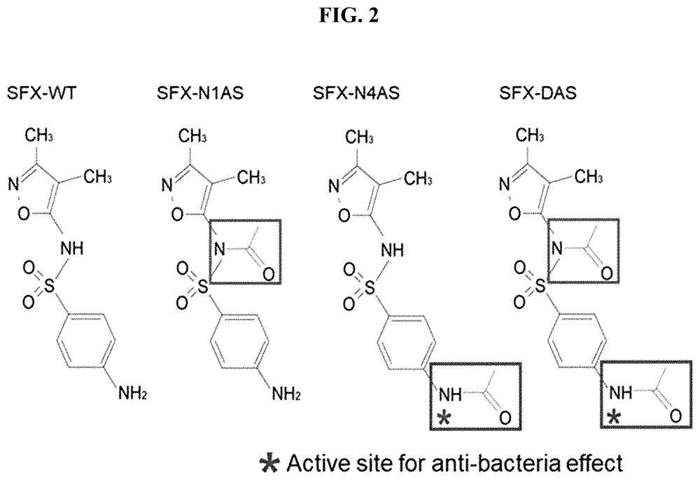

The sulfisoxazole derivatives were synthesized according to the schemes shown in FIG. 1 and FIG. 2.

1-1. Synthesis of Sulfisoxazole-N¹&N⁴-Dual Acetyl (DAS)

1 mM of SFX was dissolved in dichloromethane (DCM) and 0.21 mL of acetic anhydride was added. After warming the mixture to room temperature and adding 1:1 of hexane and ethyl acetate, pure dual acetylated SFX was obtained through purification by column chromatography on silica gel using 40-50% ethyl acetate.

1-2. Synthesis of Sulfisoxazole-N¹-Acetyl (N1AS)

After mixing 1 mM of SFX with dried tetrahydrofuran (THF), triethylamine (Et₃N) and 4-dimethylaminopyridine (DMAP) were added. Then, the mixture was cooled to −20° C. and stirred for 1 hour after adding acetic anhydride. Then, after removing the solvent using a rotary evaporator, pure N1-acetylated SFX (N1AS) was obtained through purification by column chromatography on silica gel using 50% ethyl acetate.

1-3. Synthesis of Sulfisoxazole-N⁴-Acetyl (N4AS)

Step 1: After adding DMAP and Boc-anhydride to 1 mM of SFX, the mixture was heated at 60° C. for 1 hour. Then, after cooling to room temperature and removing the solvent using a rotary evaporator, the product was purified by column chromatography on silica gel using 40% ethyl acetate.

Step 2: After adding 0.82 mM of the compound purified in the step 1 to 5 mL of DCM at 0° C., Et₃N, DMAP and acetic anhydride were added and the mixture was heated to room temperature. Then, after removing the solvent using a rotary evaporator, the product was purified by column chromatography on silica gel using 40% ethyl acetate.

Step 3: After dissolving 0.88 mM of the compound purified in the step 2 in distilled water, the mixture was heated to 100° C. for 2 hours. After cooling the mixture to room temperature and extracting with ethyl acetate, the resulting layers were combined and concentrated through drying. Then, the product was purified by column chromatography on silica gel using 60% ethyl acetate.

The synthesized sulfisoxazole derivatives were dissolved in dimethyl sulfoxide (DMSO) for use in cell experiments.

### <Example 2> Antibiotic Susceptibility of Sulfisoxazole Derivatives

In order to investigate the antibiotic susceptibility of the sulfisoxazole derivatives, minimum inhibitory concentration (MIC) assay was conducted using Mueller-Hinton agar (#225250; Difco Laboratories) according to the guidelines of the Clinical and Laboratory Standard Institute (CLSI, 2015). Staphylococcus aureus (ATCC 29213) and Escherichia coli (ATCC 25922) were used.

As a result, it was confirmed that SFX-WT showed strong antibiotic susceptibility to S. aureus and E. coli; however the antibiotic effects of SFX-N1AS were reduced significantly and the antibiotic effects of SFX-N4AS and SFX-DAS were completely disappeared, as shown in Table 1.

| | MIC (μg/ml) | | | |
|---|---|---|---|---|
| | SFX- | SFX | SFX- | SFX- |
| | WT | N1AS | N4AS | DAS |
| S.aureus | 32 | 512 | >512 | >512 |
| E.coli | 16 | 128 | >512 | >512 |

### <Example 3> Cytotoxicity of Sulfisoxazole Derivatives

After seeding MDA-MB231 cells into a 24-well plate with 1×10⁴ cells/well, the cells were stabilized by culturing for 24 hours. 24 hours later, the cells were treated with sulfisoxazole-WT (SFX-WT), sulfisoxazole-N¹-acetyl (N1AS), sulfisoxazole-N⁴-acetyl (N4AS) or sulfisoxazole-N¹&N⁴-dual acetyl (DAS) at concentrations of 100, 200, 400 or 800 μM and then cultured for 24 hours.

After the culturing, the cells were incubated for 4 hours with an MTT tetrazolium reagent. Then, cell proliferation was investigated by measuring the absorbance of reduced MTT formazan (3-(4,5-dimethylthiazol-2-yl)-2,5-diphenyl-tetrazolium bromide) at 595 nm. Docetaxel (10 μM), which is currently used as an anticancer agent, was used as a control group.

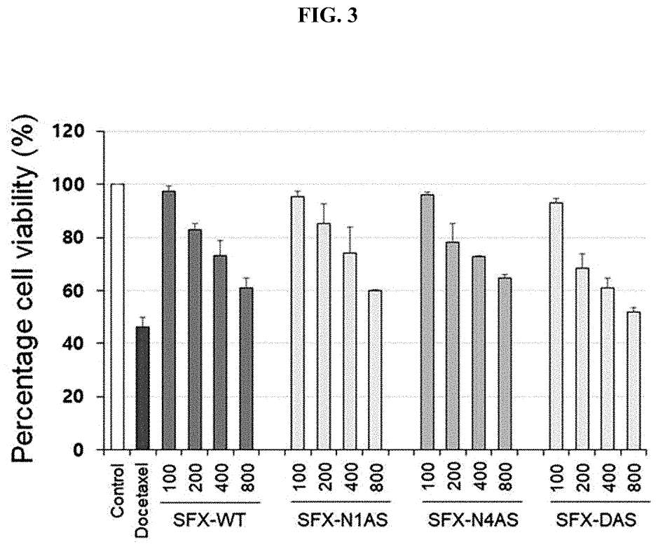

As seen from FIG. 3, it was confirmed that all the compounds SFX-WT, SFX-N1AS, SFX-N4AS and SFX-DAS exhibit cytotoxicity at 100 μM or higher.

### <Example 4> Exosome Secretion-Inhibiting Effect of Sulfisoxazole Derivatives

MDA-MB231 breast cancer cells were cultured on a 150-mm culture plate. 24 hours later, after removing the medium and washing with PBS, the cells were treated with each compound at 100 μM together with a serum-free medium and cultured for 24 hours. After discarding the medium and centrifuging at 300×g for 3 minutes, 2,500×g for 15 minutes or 10,000×g for 30 minutes, the supernatant was transferred to a fresh tube.

After filtering through a 200-nm syringe filter, exosome pellets were obtained by centrifuging at 120,000×g for 90 minutes. After redispersing the exosome pellets using PBS, the number of exosomes secreted by the cells was counted using Nano-sight LM10 (Malvern).

As seen from FIG. 4, it was confirmed that all of N1AS, N4AS and DAS strongly inhibit the secretion of exosomes as compared to SFX-WT, although the antibiotic effect was lost.

### <Example 5> Cancer Cell Migration-Inhibiting Effect of Sulfisoxazole Derivatives

After placing an insert with a pore size of 8 μm on a 24-well plate, a serum-free medium and Matrigel were coated and hardened at 37° C. for 4 hours. After adding 800 μL of 10% FBS to a lower chamber and 1×10⁴ MDA-MB231 cells to an upper chamber, sulfisoxazole or sulfadoxine was treated at 25, 50, 100 or 200 μM. 48 hours later, after removing Matrigel from the insert and washing with PBS, the lower portion of the chamber was fixed with 60% ethanol and stained with crystal violet.

In addition, after placing an insert with a pore size of 8 μm on a 24-well plate and adding 600 μL of 1% FBS to a lower chamber and 1×10⁴ MDA-MB231 cells to an upper chamber, the upper chamber was treated with SFX-WT, SFX-N1AS, SFX-N4AS or SFX DAS at 100, 200, 400 or 800 μM. 5 hours later, the insert was removed and the lower portion of the chamber was fixed with 60% ethanol and stained with crystal violet.

As seen from FIG. 5, it was confirmed that, whereas a lot of cancer cell migrated to the center in the control group, the migration of the cancer cells was inhibited in a concentration-dependent manner in the groups treated with SFX-WT, SFX-N1AS, SFX-N4AS or SFX-DAS.

While the specific aspects of the present disclosure have been described in detail above, it will be obvious to those having ordinary skill in the art that the specific description merely describes specific exemplary embodiments and the scope of the present disclosure is not limited thereby. Accordingly, the substantial scope of the present disclosure shall be defined by the appended claims and their equivalents.

1. A method of preventing or treating cancer, comprising: administering a composition comprising a compound represented by Chemical Formula 1 or a pharmaceutically acceptable salt thereof as an active ingredient to a subject in need thereof: 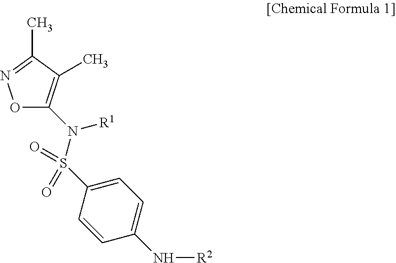
wherein
one of R¹ and R² is acetyl, and the other is any of hydrogen or acetyl.

2. The method according to claim 1, wherein the compound or a pharmaceutically acceptable salt thereof inhibits the growth and metastasis of cancer cells by inhibiting the secretion of exosomes while showing reduced antibiotic effects.

3. The method according to claim 1, wherein the cancer is selected from a group consisting of non-small-cell lung cancer, breast cancer, ovarian cancer, uterine cancer, pancreatic cancer, lung cancer, stomach cancer, liver cancer, colon cancer, skin cancer, head and neck cancer, brain cancer, laryngeal cancer, prostate cancer, bladder cancer, esophageal cancer, thyroid cancer, kidney cancer, rectal cancer, acute myeloid leukemia, chronic myeloid leukemia, acute lymphocytic leukemia, chronic lymphocytic leukemia and blood cancer.

4. The method according to claim 3, wherein the breast cancer is triple-negative breast cancer.

5. The method according to claim 1, wherein the composition is a pharmaceutical composition or a food supplement.

6. The method according to claim 5, wherein the cancer is selected from a group consisting of non-small-cell lung cancer, breast cancer, ovarian cancer, uterine cancer, pancreatic cancer, lung cancer, stomach cancer, liver cancer, colon cancer, skin cancer, head and neck cancer, brain cancer, laryngeal cancer, prostate cancer, bladder cancer, esophageal cancer, thyroid cancer, kidney cancer, rectal cancer, acute myeloid leukemia, chronic myeloid leukemia, acute lymphocytic leukemia, chronic lymphocytic leukemia and blood cancer.

7. The method according to claim 6, wherein the breast cancer is triple-negative breast cancer.

8. The method according to claim 1, wherein the treating cancer comprises inhibiting cancer metastasis.

9. A method of treating a subject having or at risk for having cancer, comprising: administering a composition comprising a compound represented by Chemical Formula 1 or a pharmaceutically acceptable salt thereof as an active ingredient to the subject: 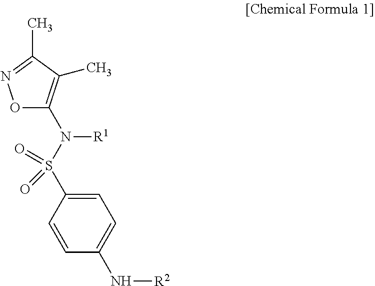
wherein
one of R¹ and R² is acetyl, and the other is any of hydrogen or acetyl.
